(19) United States
(12) Patent Application Publication
(10) Pub. No.: US 2024/0082476 A1
(43) Pub. Date: Mar. 14, 2024

(54) Medical Suction Device, Suction Pump and Method for Controlling the Same

(71) Applicant: MEDELA HOLDING AG, Baar (CH)

(72) Inventor: Hilmar Ehlert, Hergiswil (CH)

(21) Appl. No.: 18/265,181

(22) PCT Filed: Dec. 2, 2021
(86) PCT No.: PCT/EP21/83914

§ 371 Date: Jun. 2, 2023

(30) Foreign Application Priority Data
Dec. 3, 2020 (EP) 20211517.6

(51) Int. Cl. A61M 1/00

(52) U.S. Cl. CPC A61M 1/74; A61M 2205/3344

(57) ABSTRACT

A medical suction device with a medical suction pump for generating a suction pressure, which is connected at a suction side to a drainage container communicating with a suction line including a proximal end adapted to be arranged in a human body, in particular in the pericardium, a ventilation side (II) including a ventilation line the proximal end of which communicates with the distal end of the suction line and the distal end of which is provided with a controllable ventilation valve, a patient pressure sensor assigned to the ventilation side (II), and a control unit controllably connected to the ventilation valve and the patient pressure sensor. The control unit is adapted to set the ventilation valve for regulating a pressure on the ventilation side (II).

The present invention relates to the field of drainage of body fluid from the human body. In particular, the present invention is concerned with the drainage of fluid, especially blood from the pericardium, but also from the lung area (thorax).

The present invention relates in particular to a medical suction device with a medical suction pump for generating a negative pressure. A drainage container is connected to the suction side of this suction pump. The drainage container, also in the present invention, is usually detachably connected to a housing of the suction pump. The drainage container communicates with a suction line, the proximal end of which is adapted to be arranged in the human body. The medical suction device further has a ventilation side with a ventilation line, the proximal end of which communicates with the proximal end of the suction line and the distal end of which is provided with a controllable ventilation valve. Further, a patient pressure sensor is assigned to the ventilation side. The device further has a control unit controllably connected to the ventilation valve and a patient pressure sensor.

Such a medical suction device is previously known, for example, from WO 2005/061025 A1. The previously known device further has a suction pressure sensor, which is also connected to the control unit. The suction pump, too, is controllably connected to the control unit.

In the previously known state of the art, an actual pressure at the proximal end is monitored via the patient pressure sensor. If the patient pressure increases, the presence of a clogging in the region of the suction line is inferred. As a result, the suction pump is controlled to increase the suction pressure. Meanwhile, the aeration valve is opened to improve drainage of the clogging by fluid flowing out of the body in the form of a gas. The removal of the clogging in the suction line is detected by the suction pressure sensor, which is located downstream of the drainage container. In this case, the control unit prompts the suction pump to start normal suction operation, and the ventilation valve is closed.

A similar suction device is known from WO 2013/003970 A1. In this state of the art, the control unit monitors the actual suction pressure acting at the proximal end via the patient pressure sensor. The control unit is further controllably connected to a pressure sensor which is assigned to the drainage container and measures the suction pressure acting there.

Differing pressure values for the suction pressure in the drainage container and the patient pressure are assessed as a malfunction by the control unit, which thereupon emits an alarm signal. The control unit is also adapted to trigger a flushing process in which the ventilation valve and a vacuum valve controlling the actually acting suction pressure of the suction pump are opened. Thereby, air is supplied from the ventilation side and sucked at the suction side with increased suction pressure.

A similar medical suction device is known from WO 2015/010110 A1.

In current practice, the ventilation valve is opened cyclically in order to achieve an increased flow through the suction line over a certain period of time by replenishing a medium through the ventilation line.

The underlying problem of the present invention is to provide a medical suction device, a suction pump implemented therein, and a method for controlling such a suction pump, which enable suction of body fluid under improved conditions.

In order to solve the present invention according to the device, the present invention discloses a medical suction device having the features of claim 1. The medical suction pump provided therein according to the invention is indicated in claim 10.

As in the previously known state of the art, the medical suction device has a suction side and a ventilation side, each with proximal ends that can extend into the human body. In the course of therapy, the proximal end of the ventilation line and the proximal end of the suction line communicate with each other. Thus, the two lines are short-circuited. This short circuit may occur directly through the suction device, for example in a catheter thereof. Communication of the two proximal ends may also occur within the body and through the body cavity accommodating the proximal ends. It is understood that the proximal ends of the suction line and the ventilation line are usually provided immediately adjacent to each other, preferably formed to communicate with each other within a catheter.

Most preferable, the proximal end of the ventilation line and the proximal end of the suction line communicate with each other at the proximal end of a catheter. In the preferred therapy for using the suction device of the present invention, the catheter reaches into the pericardium and terminates there. Preferable, the ventilation line has a proximal end which end is at the same position with respect to the longitudinal extension of a catheter. Such catheter usually provides the ventilation line and the suction line within a single elongated body.

The present invention also has a controllable ventilation valve and a ventilation pressure sensor which are controllably connected to the control unit. However, according to the invention, the control unit is adapted to set the ventilation valve to regulate a pressure in the target area, e.g., in the pericardium or in the pleura. This also involves, for example, preventing excessive negative pressure in the target area due to an increased pumping rate to resolve the clogging by simultaneously supplying a medium to the target area until the clogging is resolved.

The present invention thus realizes a control loop the controlled variable of which is the pressure on the ventilation side, preferably measured by the patient pressure sensor. Disturbance variables of this control loop can be changes in the operating mode of the suction pump and/or a clogging within the suction line. The control unit will control the ventilation valve so that the pressure on the ventilation side corresponds to the predetermined set value.

Accordingly, applying the invention, it is possible to set a predetermined pressure in the region of the proximal end of the ventilation line and thus of the suction line, which pressure is regulated by the control unit and is controlled by actuating the ventilation valve. Accordingly, the regulation preferably occurs with the aim of maintaining a negative pressure in the target area, to avoid a drop of the negative pressure below a fixed lower limit. If, for example, the pumping rate is increased during the removal of a clogging in the suction performance and, as a result, the clogging is sucked out, the negative pressure in the target area can drop abruptly (excess vacuum). In order to prevent such an excess vacuum, the ventilation valve is opened for a short time. As a result of the control, a long-lasting excess vacuum is avoided in the target area.

In the solution according to the invention, the pressure on the ventilation side is the controlled variable of the control loop. For this purpose, the patient pressure sensor can be provided at any desired location on the ventilation side. For example, the patient pressure sensor can be arranged in the region of the proximal end of the ventilation line. Preferably, however, the patient pressure sensor is usually located extracorporeally, particularly preferably within a pump housing of the suction pump and usually behind a virus filter so that the patient pressure sensor cannot become contaminated.

The patient pressure sensor is a sensor that detects a signal corresponding to the patient pressure. This sensor may be provided proximally for insertion into the human body, particularly the pericardium. Preferably, however, the patient pressure sensor is located outside the body when the suction device is in use. The patient pressure sensor may be assigned to a line segment of the ventilation line, which preferably extends within a pump housing of the suction pump and between a ventilation interface exposed on the exterior of the pump housing and the controllable ventilation valve.

Alternatively, the patient pressure sensor can also be assigned to a separate measuring line that extends to the proximal end in order to measure the internal pressure, in particular on the ventilation side and inside the body, especially the pericardium. Such a configuration would eliminate any interference with the signal from the patient pressure sensor due to fluid and its flow in the ventilation line and thus lead to reliable measurement results with regard to the suction pressure actually acting. Accordingly, the patient pressure sensor is not necessarily a pressure sensor assigned to the ventilation line. Rather, the patient pressure sensor detects the pressure in the target area, i.e. inside the body, which can be changed by ventilation through the ventilation line.

The pump housing usually also accommodates the control unit. The same applies to a negative pressure generating means and an optionally provided suction pressure sensor, which is usually connected to a line segment of the suction line extending between a suction interface on the outside of the pump housing and the negative pressure generating means. Configured in this way, the medical suction pump can be used permanently, while the other components of the medical suction device can be consumables that are disposed of after having been used once.

However, the ventilation valve and, if necessary, also the patient pressure sensor and any suction pressure sensor provided can also be arranged outside the pump housing. The specific configuration is oriented in particular to economic considerations, taking into account sterility. The sensors can also be configured as consumables and connected to the control unit only via a datawise interface with the pump housing and data lines provided therein.

A redundant safety system can thus be realized to maintain patient safety.

The ventilation valve is preferably formed by a proportional valve. However, other valves that can be controlled to open the ventilation line are also feasible without further ado—for example, a clocked one-way valve.

The drainage container of the suction device according to the invention follows a generally known structure. The drainage container is usually connectable to the pump housing, wherein between said suction interface on the outside of the pump housing and the reservoir of the drainage container, a filter is usually provided to prevent bacteria from entering the pump housing. The drainage container is usually connected to an interface for a suction hose, which is part of the suction line and can be formed by a catheter itself or at least a hose segment of the suction line that can be connected to the catheter. A ventilation hose that can be connected to the ventilation interface of the pump housing can also be part of the catheter or connected to the catheter via a hose segment.

Preferably, the medical suction device has a connector with a catheter interface.

The proximal end of the suction line and the proximal end of the ventilation line are preferably configured by a catheter comprising for this purpose a suction lumen and a ventilation lumen which are provided parallel to each other. The catheter provides the proximal ends of the ventilation line and the suction line. As mentioned above, the proximal ends of the suction lumen and the ventilation lumen can communicate with each other within the catheter.

The catheter interface of the aforementioned connector is suitable for connecting the suction lumen and the measuring, and ventilation lumen of the catheter. The connector further has a suction hose interface for connecting a suction hose communicating with the drainage container and a ventilation hose interface communicating with the ventilation lumen and adapted for connecting a ventilation hose. When assembled, the ventilation hose typically connects the connector to the ventilation hose interface provided on the pump housing.

Thus, the possibility is created to connect a catheter individually adapted to the respective requirement to the medical suction pump according to the invention as part of the medical suction device according to the invention.

The control unit of the medical suction device according to the invention is preferably adapted to set the ventilation valve to regulate a predetermined constant pressure on the ventilation side as a set value in coordination with the control of the suction pressure by operating the suction pump. Irrespective of the disturbance variables, the control unit regulates the position of the ventilation valve such that on the ventilation side, preferably at the proximal end of the ventilation line, there is provided an at least approximately constant suction pressure within the scope of the therapeutic application, the level of which is to be kept constant by controlling the ventilation valve.

In a manner known per se, it is proposed according to the present invention to connect the control unit preferably in a controlling manner with the suction pump and a suction pressure sensor assigned to the suction side. The control unit is preferably adapted to increase the suction pressure in the event of clogging of the suction line and to control the pressure on the ventilation side with the predetermined constant pressure as a set value as a controlled variable. The detection of a clogging can thereby follow concepts known from the state of the art described above, for example by monitoring the signal of the suction pressure sensor and/or comparing the patient pressure with the suction pressure. In a contrast to the previously known solutions, according to the present invention, in the context of removing a clogging, the ventilation valve is thus not only opened completely in order to achieve a maximum replenishment of fluid via the ventilation line up to the proximal end. Rather, the ventilation valve is positioned such that the preset pressure on the ventilation side is maintained and a too high a vacuum (excess vacuum) in the target area is prevented. The previously mentioned, preferably provided measuring line is usually laid parallel to the suction line and the ventilation line and communicates proximally with the suction line and/or the ventilation line. This measuring line usually serves to detect the pressure at the proximal end of the suction device. However, in the event of clogging, the measuring line can be used as a fluid-carrying line.

In summary, it can be stated that the control due to the patient pressure sensor preferably occurs in coordination with the control of the operating parameters of the suction pump.

A controllable valve, which is controllably connected to the control unit, is preferably distally assigned to the measuring line. The controllable valve opens or closes the measuring line to the atmosphere.

Further preferably, the patient pressure sensor is assigned to the measuring line. As already discussed, the suction line is assigned the suction pressure sensor. The ventilation line is preferably assigned a further sensor that determines the pressure in the ventilation line. This further sensor is also controllably connected to the control unit.

In this further development, the control unit monitors the signals from the three sensors and can stop operation of the pump if, for example, too high a suction is detected proximally, or if a further drop in suction pressure is not detected proximally even when the negative pressure generating means is operated to set a stronger suction pressure at the proximal end. Failure of one or more of the sensors may also cause the pump to be stopped. For this purpose, the control of the pump can also act on the controllable valves to provide the best possible pressure conditions within the body, in particular the pericardium, despite a stoppage of the negative pressure generating means. The controllable valve distally assigned to the measuring line can also substitute for the ventilation valve in the event of a malfunction, for example; if the ventilation line is clogged. This can be detected, for example, by the control unit by comparing the sensor signals of the further sensor and the patient pressure sensor.

The further developments discussed and claimed for the further development of the medical suction device also apply as further developments for the medical suction pump.

The method for controlling the medical suction pump specified with a parallel aspect of the present invention is defined in claim 10. Preferred further developments of the method according to the invention are specified in dependent claims 11 and 12.

The present invention is explained below with reference to embodiments in conjunction with the drawing. Therein:

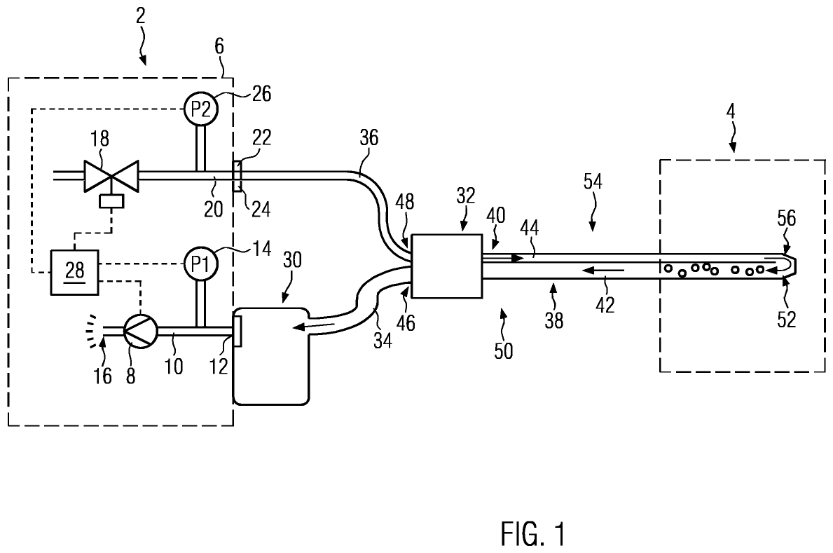

FIG. 1 shows a schematic view of a medical suction device according to the present invention with a medical suction pump according to the present invention schematically illustrated;

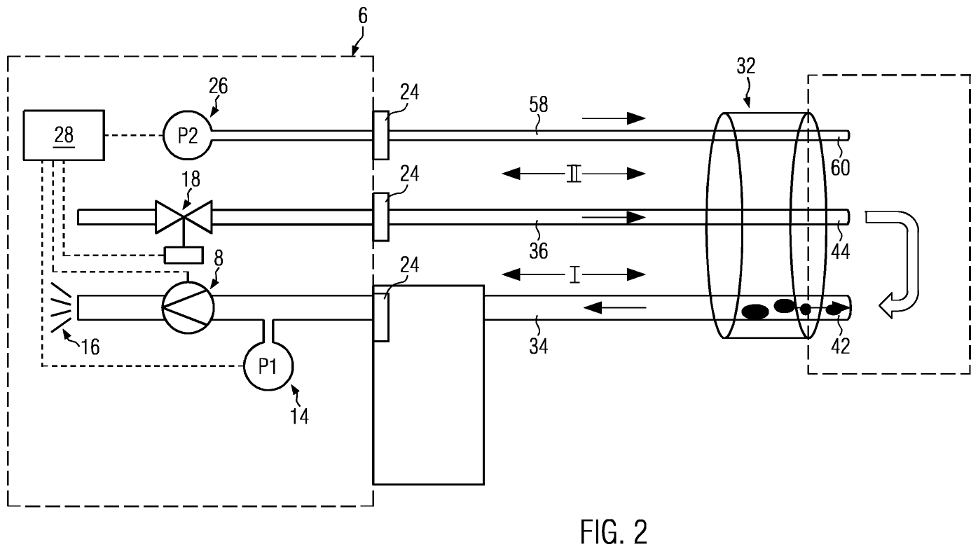

FIG. 2 shows a schematic representation of a first variant;

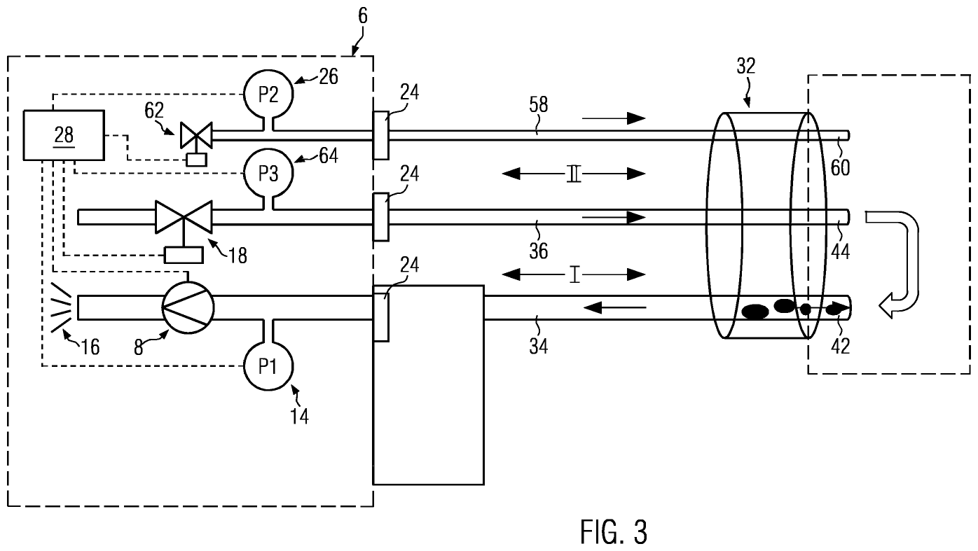

FIG. 3 shows a view according to FIG. 2 for a variation of the variant shown in FIG. 2, and

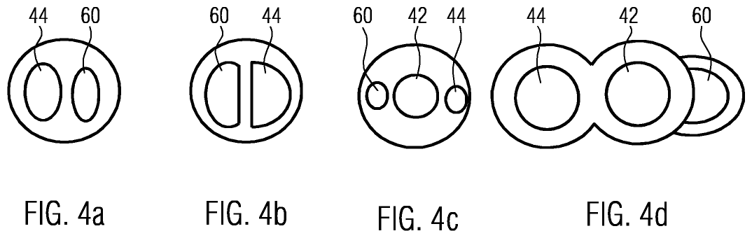

FIGS. 4a-d show various cross-sectional configurations for a hose comprising at least two lines according to the present invention.

In FIG. 1, reference sign 2 identifies the embodiment of the medical suction pump, whereas reference sign 4 is intended to identify the area of the human body.

The medical suction pump 2 has a pump housing 6 which accommodates a negative pressure generating means 8 in the form of a vacuum pump therein, which is assigned to a suction side I of the embodiment and which communicates via a suction line segment 10 with a suction interface 12 exposed on the outside of the pump housing 6. Between the negative pressure generating means 8 and the suction interface 12, a suction pressure sensor 14 is connected to the suction line segment 10. On the outlet side of the negative pressure generating means 8, an exhaust 16 is provided which typically discharges air to the atmosphere using mufflers.

The pump housing 6 further accommodates, as an example of a controllable ventilation valve 18 within the meaning of the present invention, a proportional valve which is assigned to a ventilation side II and which communicates via a ventilation line segment 20 with a ventilation interface 22 which is exposed on the outside of the pump housing 6 and which, in the present case, initially serves to connect a virus filter 24. The pressure in the ventilation line segment 20 is monitored via a patient pressure sensor 26.

Said patient pressure sensor 26 as well as the controllable ventilation valve 18, the suction pressure sensor 14, and the negative pressure generating means 8 are connected data-wise to a control unit 28, which is accommodated in the pump housing 6. Control elements for controlling the medical suction pump 2 are provided on the outside of the pump housing 6 in a manner known per se.

The components shown between the pump housing 6 and the human body 4 are consumables connected to the medical suction pump 2.

The suction interface 12 communicates with a drainage container 30, which is typically detachably connected to the pump housing 6. A suction hose 34 is located between the drainage container 30 and a connector 32. A ventilation hose 36 is located between the ventilation interface 22, specifically the virus filter 24 and the connector 32. The connector 32 serves to easily connect a catheter 38 to the interfaces 12, 22 of the pump housing 6. For this purpose, the connector 32 has a catheter interface 40 that communicates with a suction lumen 42 or a ventilation lumen 44 of the catheter 38. The catheter interface 40 is adapted for being detachably connected to the catheter 38, in particular for plug-in contacting of the catheter 38. On the opposite side of the connector 32, the connector 32 forms a suction hose interface 46 for connecting the suction hose 34 and a ventilation hose interface 48 for connecting the ventilation hose 36. The corresponding interfaces are usually formed by interfaces commonly used in medical technology, for example a Luer connection

In the embodiment shown, a suction line, identified by reference sign 50, provided on suction side I extends from the negative pressure generating means to a proximal end 52, which in the present case is formed by the proximal end of the suction lumen 42 within the catheter 38. A ventilation line, identified by reference sign 54, provides the ventilation side II and extends from the ventilation valve 18 to a proximal end, identified by reference sign 56, which is in the present case formed by the proximal end of the ventilation lumen 44. The proximal ends of the suction line 52 and the ventilation line 56 communicate with each other within the catheter 38 and, accordingly, are short-circuited to each other within the catheter 38.

When operating the embodiment according to the invention, the negative pressure generating means 8 is operated by the control unit 28. Irrespective of the actually acting suction pressure, which is monitored by the suction pressure sensor 14 and reported back to the control unit 28, the ventilation valve 18 is set to a constant suction pressure, which in the present case is monitored by the patient pressure sensor 26. According to the embodiment, this is to be kept at a constant value. If, for example, the control unit 28 detects a clogging of the suction line 50 due to an increasing pressure value on the suction side, the power of the negative pressure generating means 8 can be increased in a manner known per se, prompted by the control unit 28, in order to suck off the clogging into the drainage container 30. However, due to the control of the ventilation valve 18, the actual pressure at the proximal end 56 of the ventilation line 54 and subsequently the proximal end 52 of the suction line 50 remains constant. Irrespective of any disturbance variables and changes in the operating point of the negative pressure generating means 8, there is accordingly a constant suction pressure within the body. Damage to tissue or suction of tissue due to excessive suction pressure at the proximal end of the catheter 38 is therefore not to be feared.

FIGS. 2 and 3 illustrate an alternative embodiment and a modification thereto. Similar components compared with the embodiment shown in FIG. 1 are identified by the same reference sign. The illustration of the individual lines is schematic. Of course, these can be provided by supply lines and in a catheter, as described in principle with reference to FIG. 1.

Compared with the embodiment discussed above, the embodiment according to FIGS. 2 and 3, respectively, has a measuring line 58 which, as before, can be connected via a connector 32 and can communicate with a measuring lumen 60. Proximally, the measuring line 58 senses internal pressure in the region of the pericardium. Distally, in the embodiment shown, the patient pressure sensor 26 is provided.

In this embodiment, the measurement signal of the patient pressure sensor 26 is not influenced by the volume flow on the ventilation side II. The measuring line 58 or measuring lumen 60 only serve to measure the internal pressure in the pericardium.

In the variant according to FIG. 3, a controllable valve 62 is provided at the distal end of the measuring line 58, which is controllably connected to the control unit 28. A further sensor 64 is also connected thereto, which is assigned to the ventilation side II and measures the internal pressure in the ventilation hose 36. These two components and their controllable connection to the control unit 28 improve the safety of the device. Via the further sensor 64, a further signal can be determined which indicates the pressure conditions in the pericardium or possible clogging. During trouble-free operation, the controllable valve 62 is usually closed. In the event of a malfunction, which is determined by comparing the signals of the various pressure sensors 14, 26, 64, the control unit 28 can switch off the negative pressure generating means 8. It is also possible—for example, at the same time as the negative pressure generating means 8 is switched off—to open the further controllable valve 62, for example, in order to relieve a considerable suction pressure within the pericardium which may have been formed by closure of the ventilation side II. With this emergency switch-off, an excessively high vacuum in the pericardium is immediately lowered. A failure of one or more of the pressure sensors 14, 26, 64 can also lead to a switch-off of the medical suction pump 2, in particular of the negative pressure generating means 8. A further emergency switch-off can be stored in the control unit 28 in the event that no suction effect can be detected in the region of the pericardium and/or no significant volume flow on the suction side I despite increased output of the negative pressure generating means 8.

FIGS. 4a-d show various embodiments of hoses which realize at least two, possibly even three, of the lumens. Such hoses may, for example, be part of the catheter. In the embodiments according to FIGS. 4a, 4b, the suction lumen 42 or the suction hose 34 is realized separately. Obviously, hoses with two or three lumens can also be provided between the adapter 32 and the pump housing 6. It is understood that said pump housing 6 has an interface for the measuring line 58. As FIGS. 2 and 3 illustrate, virus filters 24 are each located at the interface for the corresponding hoses 34, 36 or lines 58, respectively.

### REFERENCE SIGN LIST

2 medical suction pump
4 human body
6 pump housing
8 negative pressure generating means
10 suction line segment
12 suction interface
14 suction pressure sensor
16 exhaust
18 ventilation valve
20 ventilation line segment
22 ventilation interface
24 virus filter
26 patient pressure sensor
28 control unit
30 drainage container
32 connector
34 suction hose
36 ventilation hose
38 catheter
40 catheter interface
42 suction lumen
44 ventilation lumen
46 suction hose interface
48 ventilation hose interface
50 suction line
52 proximal end of the suction line
54 ventilation line
56 proximal end of the ventilation line
58 measuring line
60 measuring lumen
62 controllable valve
64 further sensor
I suction side
II ventilation side

1. A medical suction device with a medical suction pump for generating a suction pressure, which is connected at a suction side (I) to a drainage container communicating with a suction line comprising a proximal end adapted to be arranged in a human body, a ventilation side (II) comprising a ventilation line the proximal end of which communicates with the distal end of the suction line and the distal end of which is provided with a controllable ventilation valve, a patient pressure sensor assigned to the ventilation side (II), and a control unit controllably connected to the ventilation valve and the patient pressure sensor, the control unit being adapted to set the ventilation valve for regulating a pressure on the ventilation side (II).

2. The medical suction device according to claim 1, the control unit adapted to set the ventilation valve to regulate a predetermined constant pressure on the ventilation side (II) as a set value.

3. The medical suction device according to claim 1, the control unit controllably communicates with the suction pump and a suction pressure sensor assigned to the suction side (I), and the control unit adapted to increase the suction pressure in the event of clogging of the suction line and to regulate the pressure on the ventilation side (II) with the predetermined constant pressure as a set value as a controlled variable.

4. The medical suction device according to claim 1, the ventilation valve formed by a proportional valve.

5. The medical suction device according to claim 1, a catheter comprising a suction lumen, the proximal end of which provides the proximal end of the suction line, and a ventilation lumen, the proximal end of which provides the proximal end (56) of the ventilation line.

6. The medical suction device according to claim 5, further comprising a connector with a catheter interface for connecting the suction lumen and the ventilation lumen of the catheter, a suction hose interface communicating with the suction lumen for connecting a suction hose communicating with the drainage container, and a ventilation hose interface communicating with the ventilation lumen for connecting a ventilation hose connected to a ventilation interface of the suction pump.

7. The medical suction device according to claim 1, a measuring line laid parallel to the suction line and the ventilation line and communicating proximally with at least one of the suction line or the ventilation line.

8. The medical suction device according to claim 7, further comprising a controllable valve controllably connected to the control unit distally assigned to the measuring line.

9. The medical suction device according to claim 7, the patient pressure sensor being assigned to the measuring line, a suction pressure sensor (14) being assigned to the suction line, and further comprising a further sensor determining the pressure in the ventilation line assigned to the ventilation line, and wherein the patient pressure sensor, the suction pressure sensor, and the further sensor are controllably connected to the control unit.

10. A medical suction pump for generating a suction pressure, with a pump housing in which a negative pressure generating means is accommodated, the suction side (I) of which is connected to a suction interface to which a drainage container which can be detachably connected to the pump housing can be connected, and a controllable ventilation valve, the ventilation side (II) of which is connected to a ventilation interface exposed on the outside of the pump housing, to which a ventilation hose can be connected, wherein a patient pressure sensor is provided between the ventilation valve and the ventilation interface, and with a control unit which is controllably connected to the ventilation valve and the patient pressure sensor, and the control unit is adapted to set the ventilation valve for regulating a pressure on the ventilation side (II).

11. A method for controlling a medical suction pump for generating a suction pressure on a suction side (I) comprising a suction line (50) which has a proximal end adapted to be arranged in a human body, and the distal end of which communicates with the suction pump via a drainage container, wherein on a ventilation side (II) a ventilation line is provided, the proximal end of which communicates with the proximal end of the suction line, wherein in the method the pressure on the ventilation side (II) is regulated.

12. The method according to claim 11, wherein the regulation occurs with a predetermined constant pressure on the ventilation side (II) as a set value.

13. The method according to claim 11, wherein in case of clogging of the suction line, the suction pressure is increased and the pressure on the ventilation side is regulated with the predetermined constant pressure as a set value.
